(19) United States
(12) Patent Application Publication
(10) Pub. No.: US 2026/0198853 A1
(43) Pub. Date: Jul. 16, 2026

(54) PHYSIOLOGIC DETECTION APPARATUS HAVING IMMERSION CONFIGURATION AND SIGNAL DETECTION DEVICE

(71) Applicant: ZENTAN TECHNOLOGY CO., LTD., NEW TAIPEI CITY (TW)

(72) Inventors: TONG-PIE CHEN, NEW TAIPEI CITY (TW); YU-JU CHEN, NEW TAIPEI CITY (TW)

(21) Appl. No.: 19/023,972

(22) Filed: Jan. 16, 2025

(51) Int. Cl. A61B 5/00

(52) U.S. Cl. CPC A61B 5/6801; A61B 5/6831; A61B 2562/18

(57) ABSTRACT

A physiologic detection apparatus having an immersion configuration and a signal detection device. The signal detection device includes a wearable carrier, two insulating layers disposed on the wearable carrier, a conductive rubber layer sandwiched between the two insulating layers, a first buckle assembled to the two insulating layers, a second buckle engaged with the first buckle, and a sealing gasket that is arranged between the first buckle and the second buckle. A part of the first buckle protrudes from the wearable carrier and one of the two insulating layers adjacent thereto, and is engaged with the second buckle and is surrounded by the sealing gasket. The sealing gasket abuts against the wearable carrier and one of the two insulating layers adjacent thereto.

### FIELD OF THE DISCLOSURE

The present disclosure relates to an immersion detection apparatus, and more particularly to a physiologic detection apparatus having an immersion configuration and a signal detection device.

### BACKGROUND OF THE DISCLOSURE

When a conventional physiological signal detection device is worn by a user and is immersed in water, the conventional physiological signal detection device may have a detection error due to an internal signal transmission thereof being affected by the water, such that a scope of application of the conventional physiological signal detection device is limited.

### SUMMARY OF THE DISCLOSURE

In response to the above-referenced technical inadequacies, the present disclosure provides a physiologic detection apparatus having an immersion configuration and a signal detection device for effectively improving on the issues associated with conventional physiological signal detection devices.

In order to solve the above-mentioned problems, one of the technical aspects adopted by the present disclosure is to provide a physiologic detection apparatus having an immersion configuration, which includes a signal detection device and a signal transceiver. The signal detection device includes a wearable carrier, a first insulating layer, a conductive rubber layer, a second insulating layer, a first buckle, a sealing gasket, and a second buckle. The wearable carrier has a first surface and a second surface that is opposite to the first surface. The wearable carrier has a thru-hole. The first insulating layer is disposed on the first surface of the wearable carrier and has a sealing hole that corresponds in position to the thru-hole. The conductive rubber layer is disposed on the first insulating layer. The second insulating layer is disposed on the conductive rubber layer and has a detection opening. The conductive rubber layer is sealed and sandwiched between the first insulating layer and the second insulating layer. The conductive rubber layer has a contact portion that is exposed from and protrudes from the detection opening. The first buckle includes a sheet and a pillar. The sheet is embedded in-between the first insulating layer and the second insulating layer and is in contact with the conductive rubber layer. The pillar extends from the sheet and passes through the sealing hole and the thru-hole, and a connection between the pillar and the sealing hole of the first insulating layer has an interference-fit configuration. The sealing gasket includes an inner ring and an outer ring. The inner ring is arranged in the thru-hole and abuts against a part of the first insulating layer that is located outside of the sealing hole. The pillar is arranged inside of the inner ring. The outer ring extends from the inner ring and abuts against the second surface of the wearable carrier. The second buckle is engaged with the pillar of the first buckle and abuts against the outer ring so as to enable the outer ring to be sandwiched between the second buckle and the wearable carrier. The signal transceiver has an insulating portion and a conductive portion that is arranged inside of the insulating portion. The insulating portion abuts against the outer ring of the sealing gasket, and the conductive portion is detachably connected to the first buckle and the second buckle of the signal detection device. When the physiologic detection apparatus is worn by a user and is immersed in liquid, the contact portion of the conductive rubber layer is configured to receive a physiologic signal from the user, and the first buckle and the second buckle are configured to transmit the physiologic signal from the contact portion to the signal transceiver.

In order to solve the above-mentioned problems, another one of the technical aspects adopted by the present disclosure is to provide a signal detection device, which includes a wearable carrier, a first insulating layer, a conductive rubber layer, a second insulating layer, a first buckle, a sealing gasket, and a second buckle. The wearable carrier has a first surface and a second surface that is opposite to the first surface. The wearable carrier has a thru-hole. The first insulating layer is disposed on the first surface of the wearable carrier and has a sealing hole that corresponds in position to the thru-hole. The conductive rubber layer is disposed on the first insulating layer. The second insulating layer is disposed on the conductive rubber layer and has a detection opening. The conductive rubber layer is sealed and sandwiched between the first insulating layer and the second insulating layer. The conductive rubber layer has a contact portion that is exposed from and protrudes from the detection opening. The first buckle includes a sheet and a pillar. The sheet is embedded in-between the first insulating layer and the second insulating layer and is in contact with the conductive rubber layer. The pillar extends from the sheet and passes through the sealing hole and the thru-hole, and a connection between the pillar and the sealing hole of the first insulating layer has an interference-fit configuration. The sealing gasket includes an inner ring and an outer ring. The inner ring is arranged in the thru-hole and abuts against a part of the first insulating layer that is located outside of the sealing hole. The pillar is arranged inside of the inner ring. The outer ring extends from the inner ring and abuts against the second surface of the wearable carrier. The second buckle is engaged with the pillar of the first buckle and abuts against the outer ring so as to enable the outer ring to be sandwiched between the second buckle and the wearable carrier.

Therefore, the physiologic detection apparatus and the signal detection device in the present disclosure are each provided with the detection module having a specific structural arrangement (that can further be in cooperation with the insulating portion of the signal transceiver), thereby preventing a signal transmission of the pillar of the first buckle from being affected by the liquid. Accordingly, a detection error generated from the signal detection device can be avoided for effectively expanding a scope of application of the physiologic detection apparatus (or the signal detection device).

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic perspective view of a physiologic detection apparatus having an immersion configuration according to a first embodiment of the present disclosure;

FIG. 2 is a schematic exploded view of FIG. 1;

FIG. 3 is a schematic perspective view showing a part of a signal detection device according to the first embodiment of the present disclosure;

FIG. 4 is a schematic exploded view of FIG. 3;

FIG. 5 is a schematic exploded view of FIG. 3 from another angle of view;

FIG. 6 is a schematic enlarged view showing a first buckle, a sealing washer, and a second buckle of FIG. 4;

FIG. 7 is a schematic enlarged view showing the first buckle, the sealing washer, and the second buckle of FIG. 5;

FIG. 8 is a schematic cross-sectional view taken along line VIII-VIII of FIG. 1;

FIG. 9 is a schematic cross-sectional view taken along line IX-IX of FIG. 3; and

FIG. 10 is a schematic perspective view showing a part of the signal detection device according to a second embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

### First Embodiment

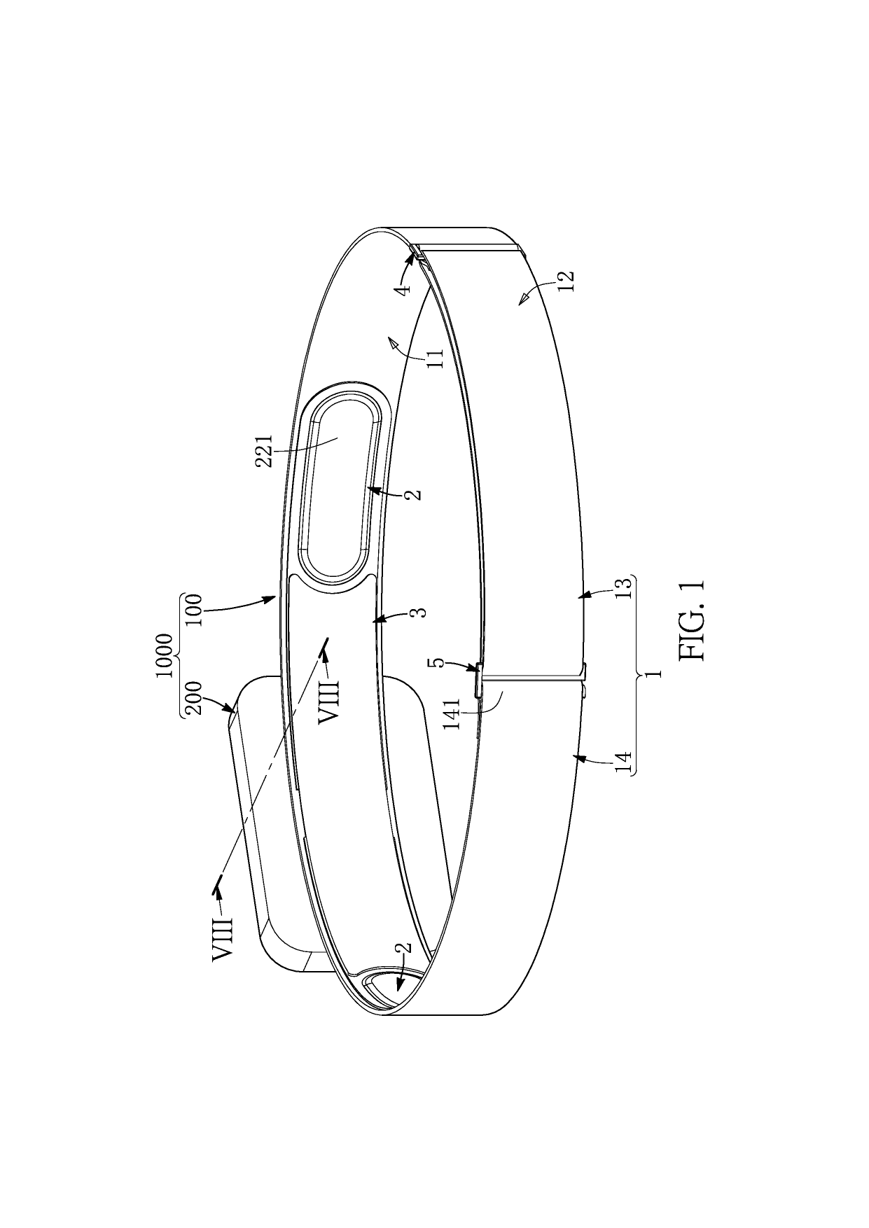

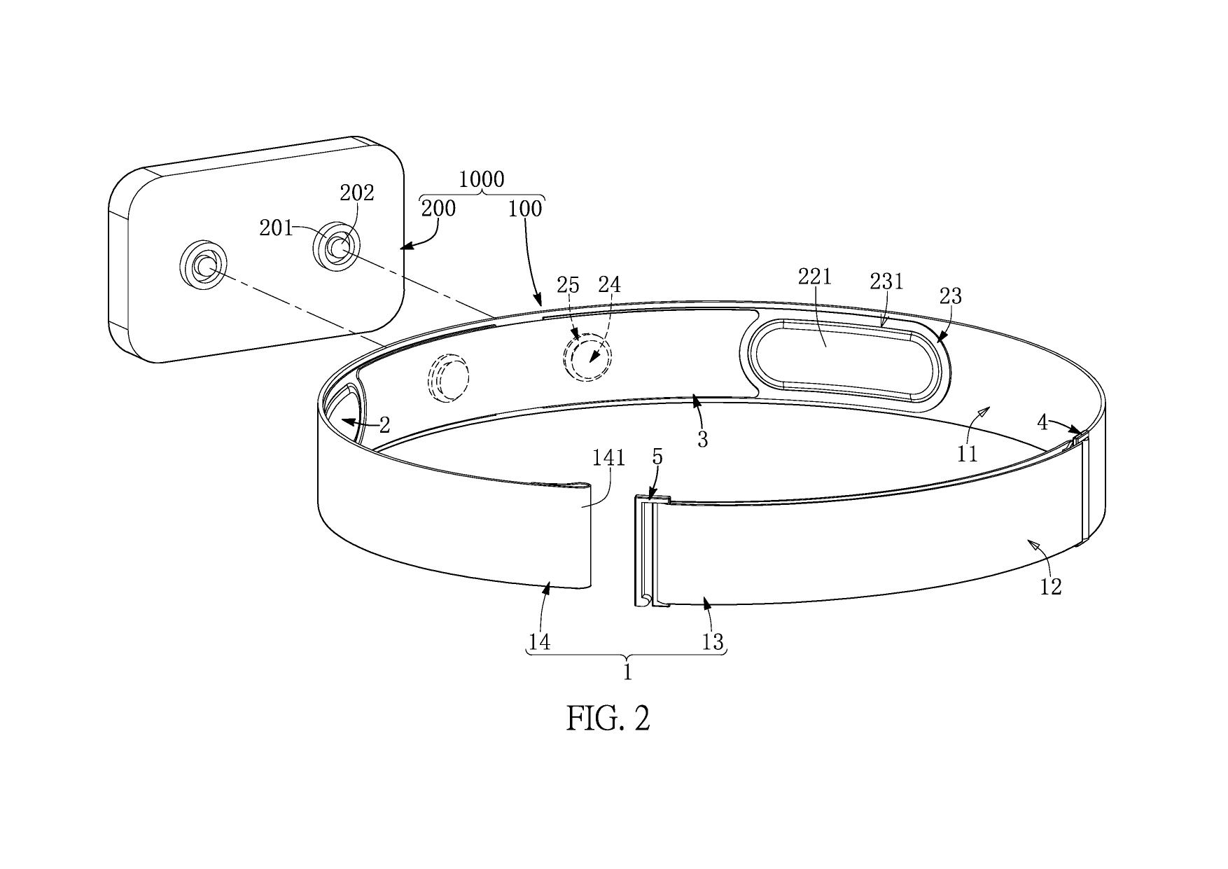

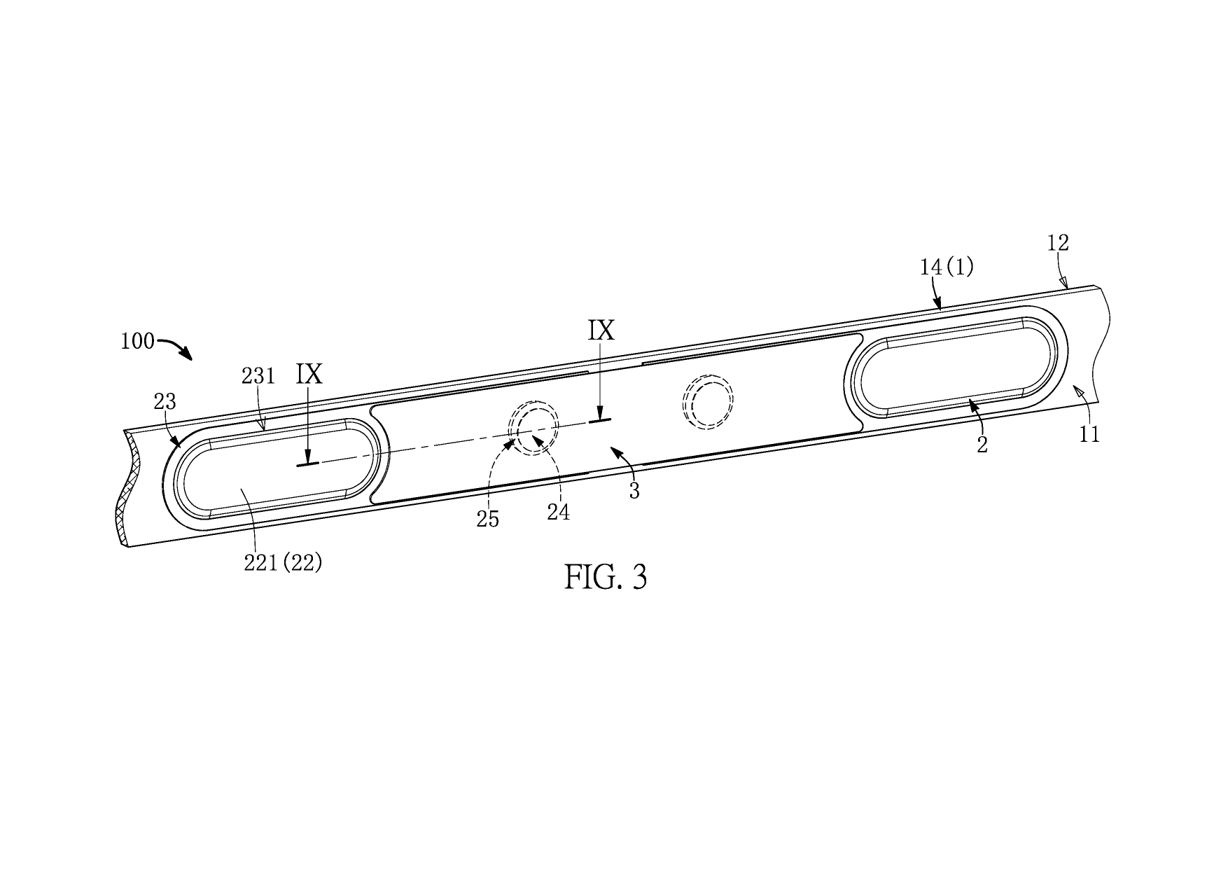

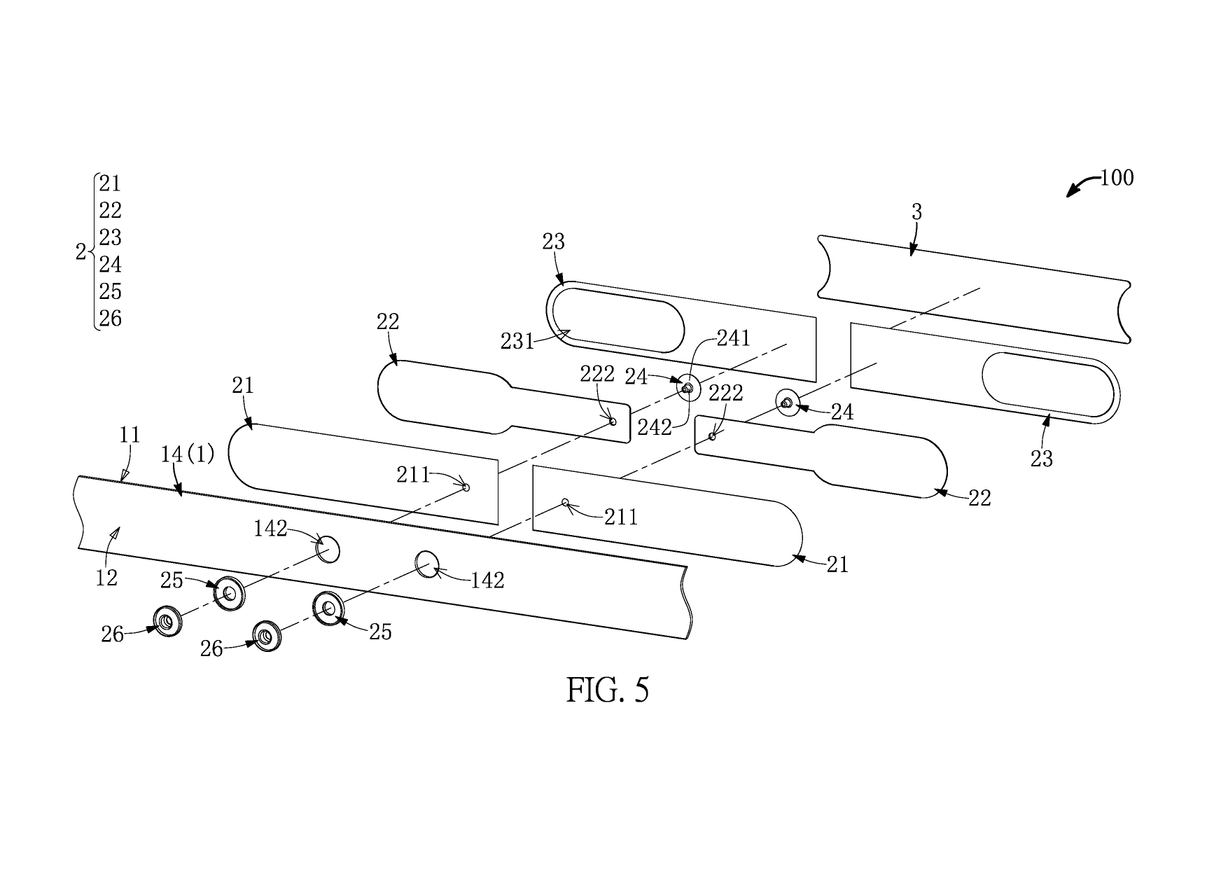

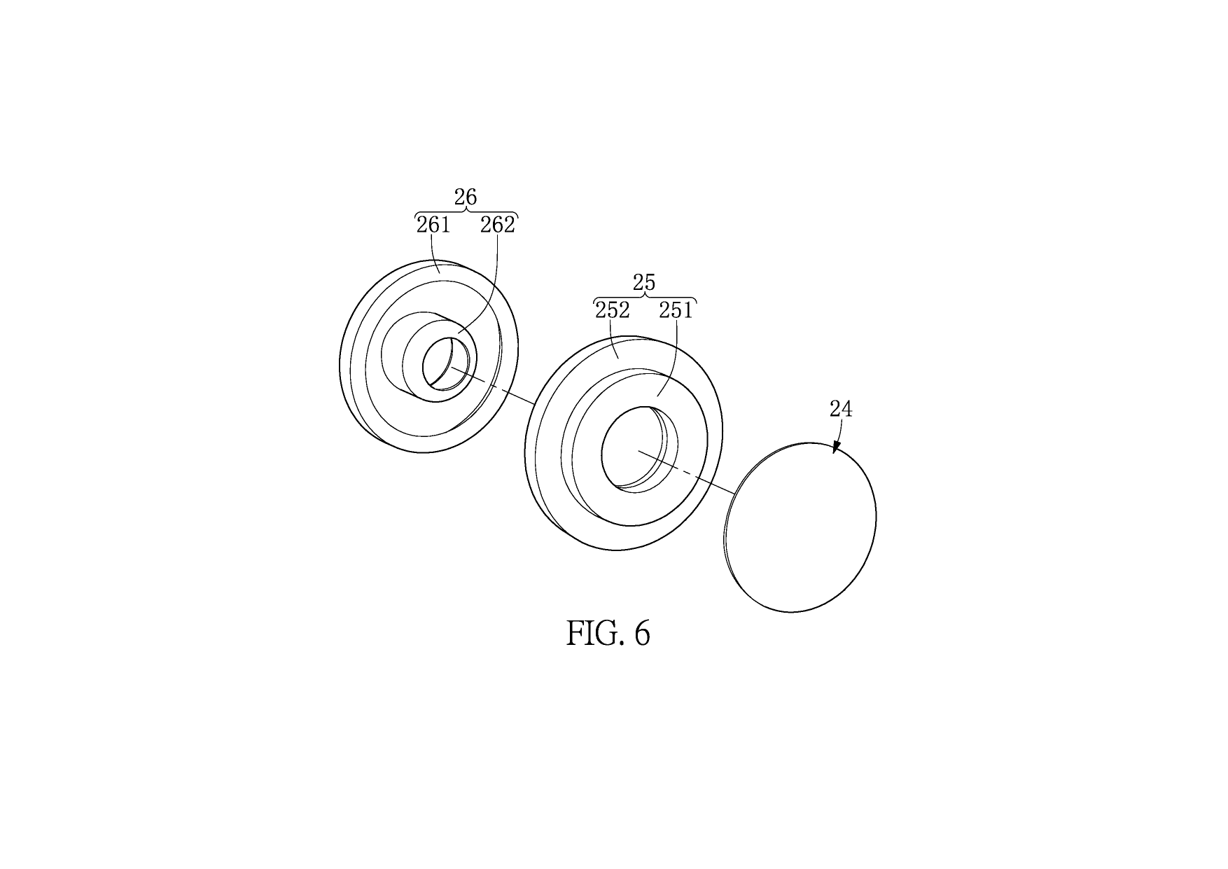

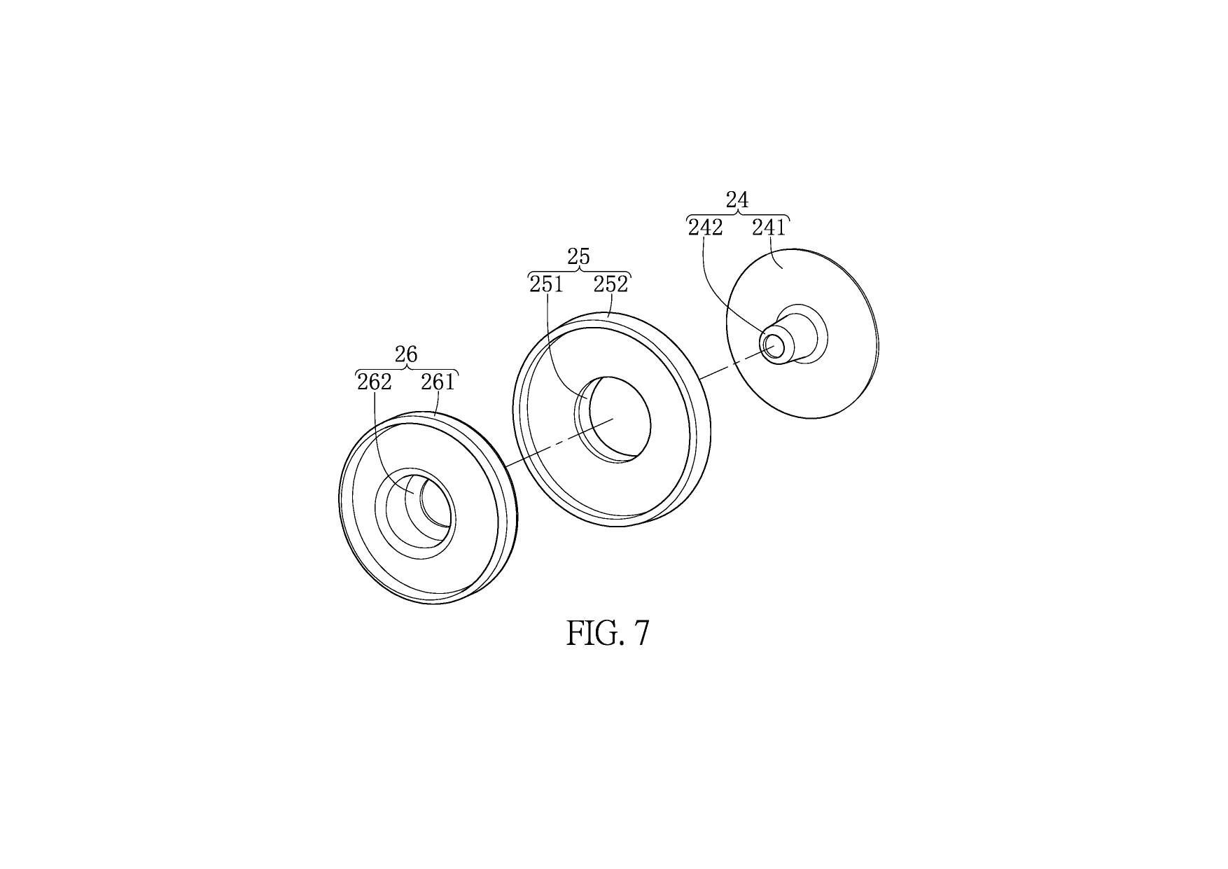

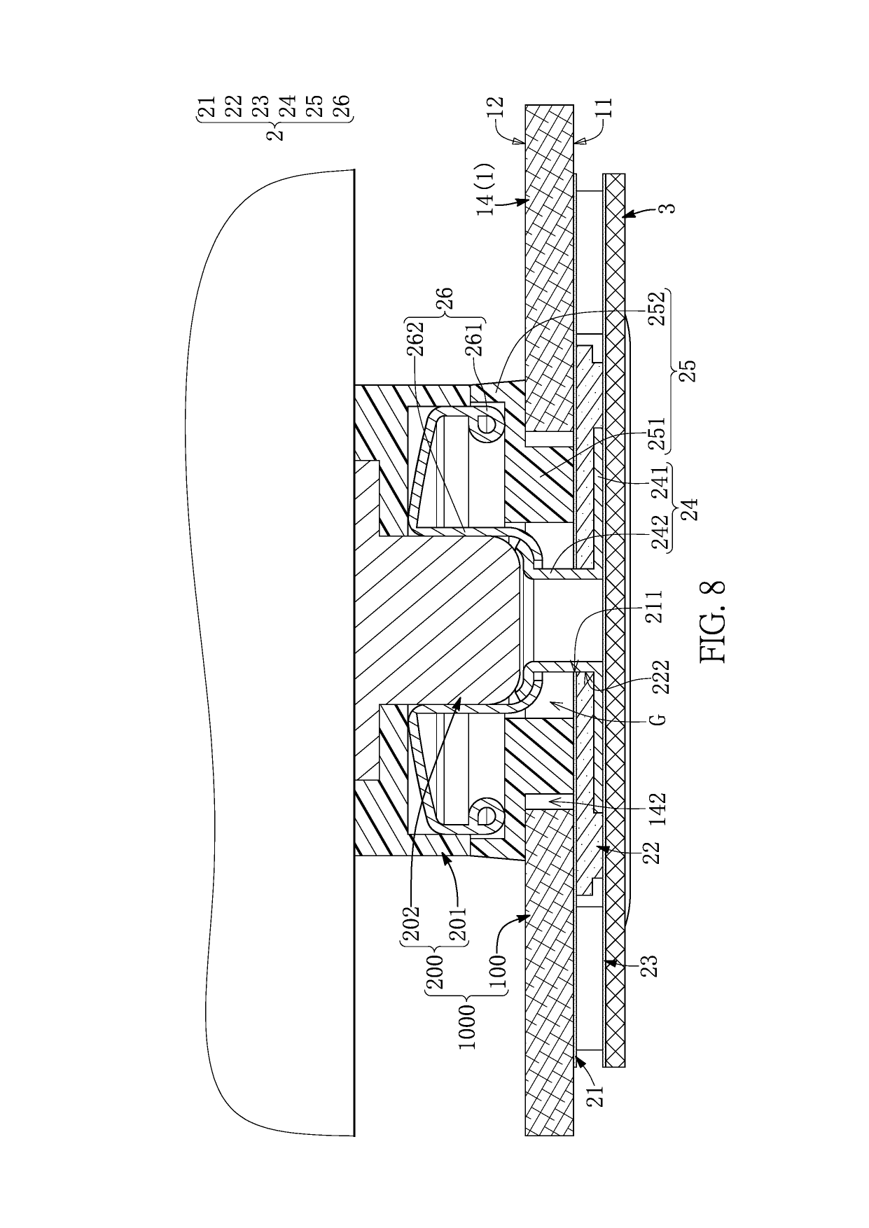

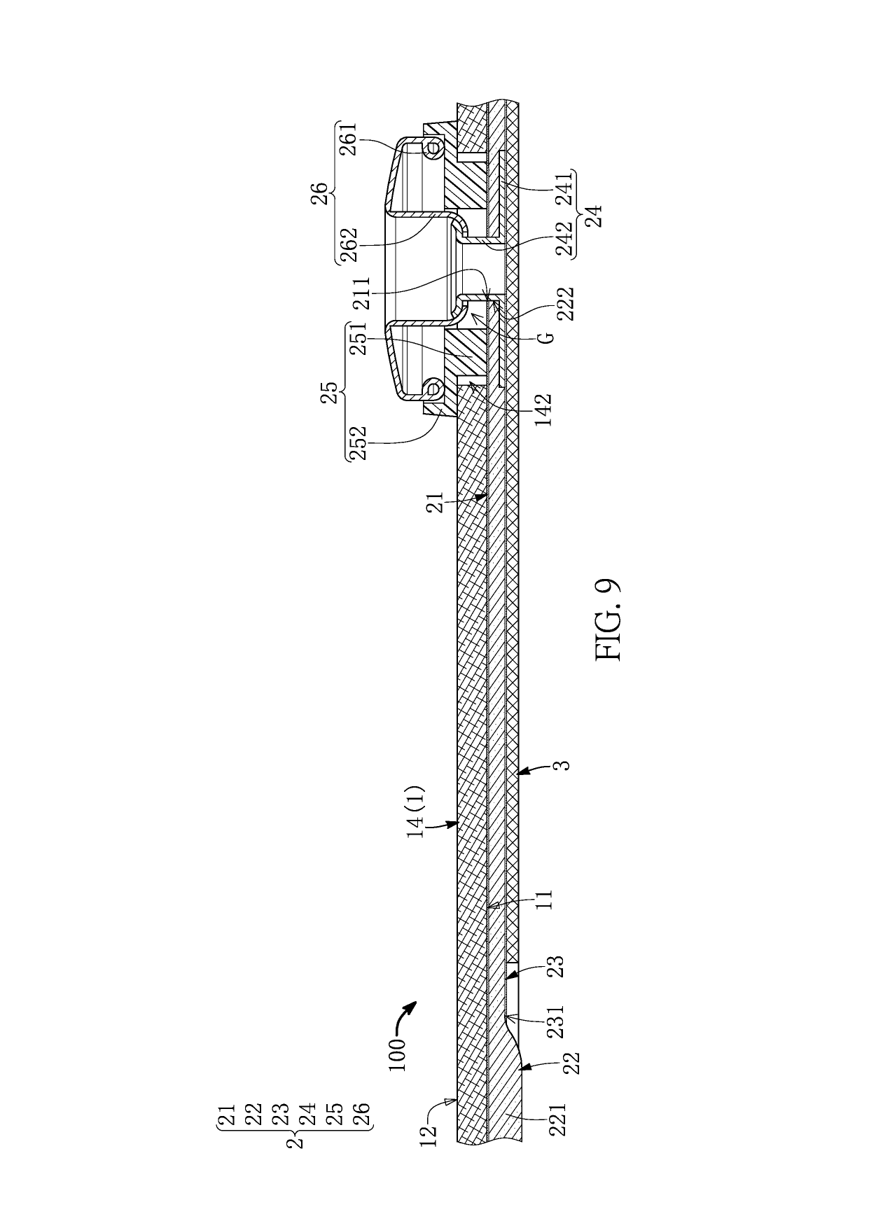

Referring to FIG. 1 to FIG. 9, a first embodiment of the present disclosure is provided. As shown in FIG. 1 to FIG. 3, the present embodiment provides a physiologic detection apparatus 1000 having an immersion configuration, which includes a signal detection device 100 and a signal transceiver 200 that is detachably connected to the signal detection device 100. It should be noted that the physiologic detection apparatus 1000 is limited to being worn by a user and to be immersed in liquid, so that any physiologic detection apparatus being unable to be immersed in liquid is different from the physiologic detection apparatus 1000 provided by the present embodiment.

Moreover, the signal detection device 100 in the present embodiment is described in cooperation with the signal transceiver 200, but the present disclosure is not limited thereto. For example, in other embodiments of the present disclosure not shown in the drawings, the signal detection device 100 can be independently used (e.g., sold) or can be used in cooperation with other components.

In the present embodiment, the signal detection device 100 includes a wearable carrier 1, two detection modules 2 disposed on the wearable carrier 1 and spaced apart from each other, a waterproof cloth 3 disposed on the two detection modules 2, an adjustment metal ring 4 assembled to the wearable carrier 1, and a metal hook 5 that is assembled to the wearable carrier 1. The wearable carrier 1 has a first surface 11 and a second surface 12 that is opposite to the first surface 11, and the two detection modules 2 are preferably disposed on the first surface 11 of the wearable carrier 1.

The wearable carrier 1 is elongated, and includes an elastic adjustment segment 13 and a detection segment 14 that is connected to an end of the elastic adjustment segment 13. The elastic adjustment segment 13 is configured to change or adjust a wearable length of the wearable carrier 1 for being applied to different wearing requirements. The detection segment 14 is provided with the two detection modules 2 and the waterproof cloth 3 disposed thereon, and the signal transceiver 200 is detachably connected to the two detection modules 2 of the signal detection device 100, but the present disclosure is not limited thereto. For example, in other embodiments of the present disclosure not shown in the drawings, the wearable carrier 1 can be not elongated; or, a quantity of the detection modules 2 can be one or more than one for being in cooperation with the wearable carrier 1 according to practical requirements.

Specifically, the adjustment metal ring 4 is arranged between the elastic adjustment segment 13 and the detection segment 14 so as to enable the elastic adjustment segment 13 to change the wearable length of the wearable carrier 1 through the adjustment metal ring 4. Moreover, the detection segment 14 further has an assembling portion 141 arranged away from the elastic adjustment segment 13, the metal hook 5 is assembled to the elastic adjustment segment 13, and the metal hook 5 is detachably engaged with the assembling portion 141 of the detection segment 14 so as to enable the wearable carrier 1 to have a loop shape. The adjustment metal ring 4 and the metal hook 5 are substantially located at two opposite ends of the elastic adjustment segment 13, thereby providing a firm structural strength.

As the two detection modules 2 in the present embodiment are of substantially the same structure and are in a substantially mirror symmetrical arrangement, the following description discloses the structure of just one of the two detection modules 2 and a corresponding part of the wearable carrier 1 (e.g., a part of the detection segment 14) for the sake of brevity, and then discloses the connection relationship of the two detection modules 2, but the present disclosure is not limited thereto. For example, in other embodiments of the present disclosure not shown in the drawings, the two detection modules 2 can be of different structures.

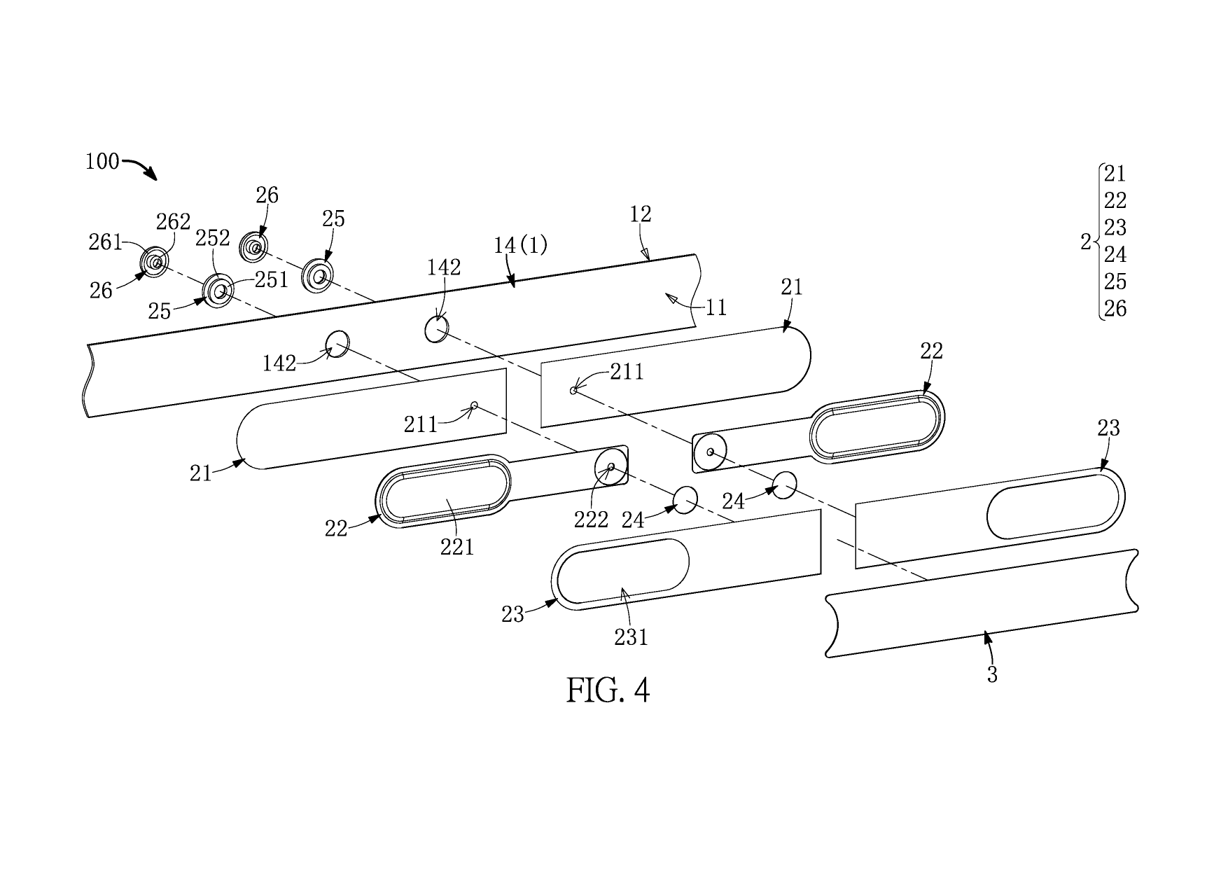

As shown in FIG. 4 to FIG. 9, the detection module 2 in the present embodiment includes a first insulating layer 21 disposed on the first surface 11, a conductive rubber layer 22 disposed on the first insulating layer 21, a second insulating layer 23 disposed on the conductive rubber layer 22, a first buckle 24 assembled in-between the first insulating layer 21 and the second insulating layer 23, a sealing gasket 25 corresponding in position to the first buckle 24, and a second buckle 26 that is assembled to the first buckle 24 and the sealing gasket 25.

The wearable carrier 1 (e.g., the detection segment 14) has a thru-hole 142, the first insulating layer 21 has a sealing hole 211 corresponding in position to the thru-hole 142, and an aperture of the sealing hole 211 is less than an aperture of the thru-hole 142. Moreover, a periphery of the second insulating layer 23 is gaplessly connected to a periphery of the first insulating layer 21, and the second insulating layer 23 has a detection opening 231 arranged away from the sealing hole 211.

The conductive rubber layer 22 is sealed and sandwiched between the first insulating layer 21 and the second insulating layer 23, and the conductive rubber layer 22 has a contact portion 221 that is exposed from and protrudes from the detection opening 231. In other words, each of the first insulating layer 21 and the second insulating layer 23 is gaplessly connected to the conductive rubber layer 22, so that any liquid cannot flow into a region between the first insulating layer 21 and the second insulating layer 23 by flowing along the contact portion 221 (or passing through the detection opening 231).

Moreover, an outer surface of the contact portion 221 has an anti-slip texture, such that when the physiologic detection apparatus 1000 is worn by the user, the anti-slip texture is configured to prevent the contact portion 221 from sliding relative to the user. In addition, the conductive rubber layer 22 in the present embodiment further has an opening 222 corresponding in position to the sealing hole 211.

The first buckle 24 and the second buckle 26 in the present embodiment are a pair of rivet buckles cooperated with each other; for example, the first buckle 24 is a male buckle, and the second buckle 26 is a female buckle that is in cooperation with the first buckle 24, but the present disclosure is not limited thereto. The first buckle 24 has a sheet 241 and a pillar 242 that extends from the sheet 241. The sheet 241 is embedded in-between the first insulating layer 21 and the second insulating layer 23 and is in contact with the conductive rubber layer 22, and the pillar 242 passes through the sealing hole 211 and the thru-hole 142.

In the present embodiment, the sheet 241 is sandwiched between the conductive rubber layer 22 and the second insulating layer 23, and the pillar 242 sequentially passes through the opening 222, the sealing hole 211, and the thru-hole 142. Moreover, a connection between the pillar 242 and the sealing hole 211 of the first insulating layer 21 preferably has an interference-fit configuration, thereby preventing the liquid from flowing through the sealing hole 211.

The sealing gasket 25 in the present embodiment is integrally formed as a single one-piece structure, and the sealing gasket 25 is assembled and arranged between the first buckle 24 and the second buckle 26 for jointly providing a waterproof property that the first buckle 24 and the second buckle 26 fail to possess when being assembled with each other.

The sealing gasket 25 includes an inner ring 251 and an outer ring 252 that extends from the inner ring 251. The inner ring 251 is arranged in the thru-hole 142 and abuts against a part of the first insulating layer 21 that is located outside of the sealing hole 211, thereby preventing the liquid from flowing to the sealing hole 211 (or the pillar 242) along a region between the first insulating layer 21 and the wearable carrier 1. Moreover, the pillar 242 is arranged inside of the inner ring 251, and the pillar 242 and the inner ring 251 jointly define an annular gap G therebetween. The outer ring 252 outwardly extends from a top edge of the inner ring 251, and abuts against the second surface 12 of the wearable carrier 1.

The second buckle 26 is engaged with the pillar 242 of the first buckle 24 and abuts against the outer ring 252 so as to enable the outer ring 252 to be sandwiched between the second buckle 26 and the wearable carrier 1, thereby preventing the liquid from flowing to the sealing hole 211 (or the pillar 242) along a region between the outer ring 252 and the second buckle 26. Specifically, the second buckle 26 includes a crimping ring 261 and a fixing portion 262 that extends from an inner edge of the crimping ring 261. The crimping ring 261 abuts against the outer ring 252, and the fixing portion 262 passes through the thru-hole 142 (e.g., the annular gap G) and is fixed to the pillar 242.

The structure of the detection module 2 is described in the above description, and the following description describes the connection relationship between the two detection modules 2. Specifically, the first buckle 24, the sealing gasket 25, and the second buckle 26 of one of the two detection modules 2 are respectively arranged adjacent to the first buckle 24, the sealing gasket 25, and the second buckle 26 of another one of the two detection modules 2. Moreover, the contact portions 221 are arranged on two sides of the two detection modules 2 away from each other, and the waterproof cloth 3 is disposed on the second insulating layers 23 of the two detection modules 2 and is located between the contact portions 221 of the two detection modules 2.

The signal transceiver 200 has an insulating portion 201 and a conductive portion 202 that is arranged inside of the insulating portion 201. The insulating portion 201 abuts against the outer ring 252 of the sealing gasket 25, thereby preventing the liquid from flowing to the pillar 242 along a region between the insulating portion 201 and the outer ring 252. Moreover, the conductive portion 202 is detachably connected to the first buckle 24 and the second buckle 26 of the signal detection device 100. In other words, the conductive portion 202 of the signal transceiver 200 is detachably connected to the first buckle 24 and the second buckle 26 of each of the two detection modules 2 of the signal detection device 100.

In summary, the physiologic detection apparatus 1000 in the present embodiment is provided with the detection module 2 having a specific structural arrangement (that can further be in cooperation with the insulating portion 201 of the signal transceiver 200), thereby preventing a signal transmission of the pillar 242 of the first buckle 24 from being affected by the liquid. Accordingly, a detection error generated from the signal detection device 100 can be avoided.

In other words, when the physiologic detection apparatus 1000 is immersed in the liquid, the liquid does not flow to the pillar 242 of the first buckle 24. Moreover, when the physiologic detection apparatus 1000 is worn by the user and is immersed in the liquid, the contact portion 221 of the conductive rubber layer 22 is configured to receive a physiologic signal from the user, and the first buckle 24 and the second buckle 26 are configured to transmit the physiologic signal from the contact portion 221 to the signal transceiver 200.

### Second Embodiment

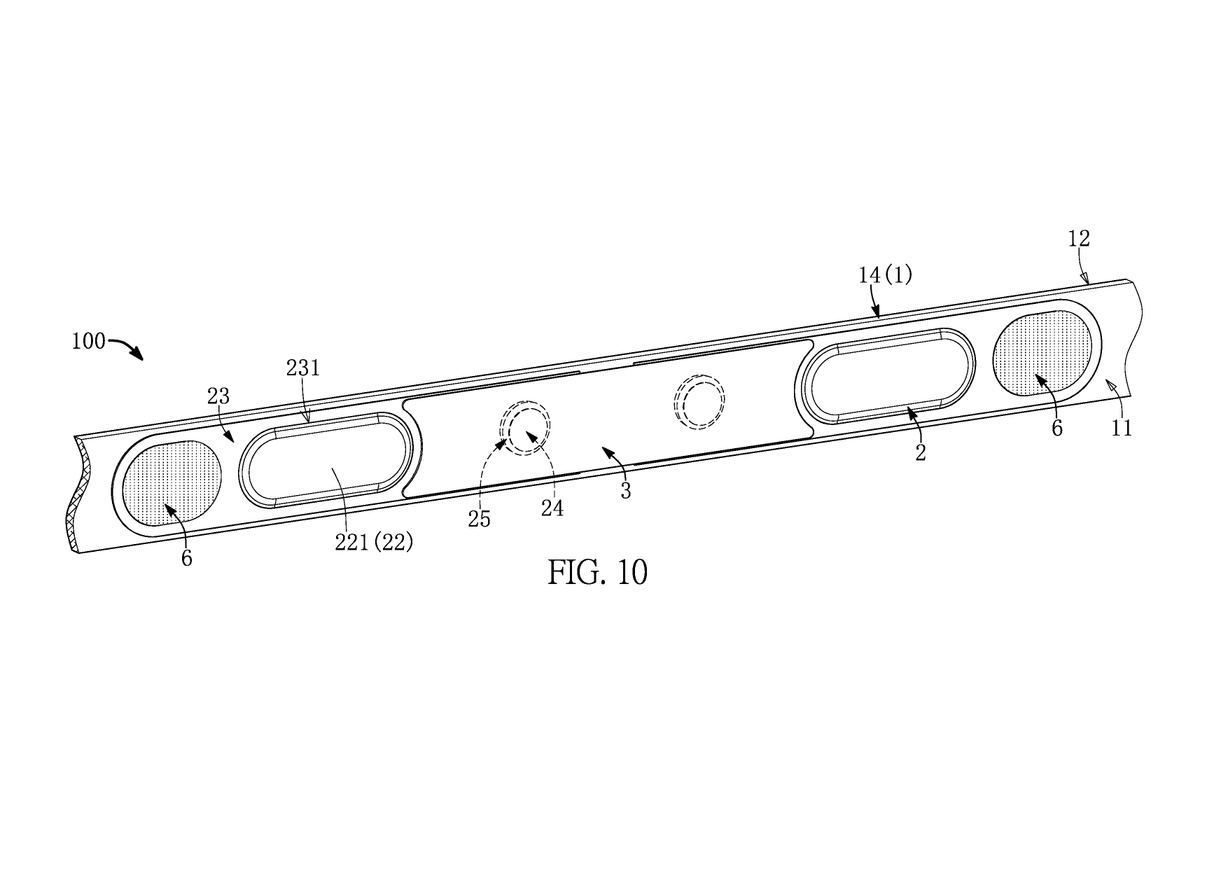

Referring to FIG. 10, a second embodiment of the present disclosure, which is similar to the first embodiment of the present disclosure, is provided. For the sake of brevity, descriptions of the same components in the first and second embodiments of the present disclosure will be omitted herein, and the following description only discloses different features between the first and second embodiments.

In the present embodiment, the signal detection device 100 further includes an antistatic layer 6 that is formed on the first surface 11 and that is arranged adjacent to the contact portion 221 of the conductive rubber layer 22. In other words, the antistatic layer 6 is arranged away from the first buckle 24. In addition, the signal detection device 100 can include two antistatic layers 5 that are disposed on the wearable carrier 1 and that are respectively located at two opposite outer sides of the two detection modules 2, but present disclosure is not limited thereto.

Accordingly, the signal detection device 100 in the present embodiment is provided with the antistatic layer 6, such that an accuracy and a stability of detection of the signal detection device 100 can be increased for meeting a broader range of requirements.

### Beneficial Effects of the Embodiments

In conclusion, the physiologic detection apparatus and the signal detection device in the present disclosure are each provided with the detection module having a specific structural arrangement (that can be further in cooperation with the insulating portion of the signal transceiver), thereby preventing a signal transmission of the pillar of the first buckle from being affected by the liquid. Accordingly, a detection error generated from the signal detection device can be avoided for effectively expanding a scope of application of the physiologic detection apparatus (or the signal detection device).

Moreover, the physiologic detection apparatus and the signal detection device in the present disclosure are each provided with the contact portion having a protruding shape, thereby enabling the contact portion to stably contact a to-be-detected portion of the user.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A physiologic detection apparatus having an immersion configuration, comprising:
a signal detection device including:
a wearable carrier having a first surface and a second surface that is opposite to the first surface, wherein the wearable carrier has a thru-hole;
a first insulating layer disposed on the first surface of the wearable carrier and having a sealing hole that corresponds in position to the thru-hole;
a conductive rubber layer disposed on the first insulating layer;
a second insulating layer disposed on the conductive rubber layer and having a detection opening, wherein the conductive rubber layer is sealed and sandwiched between the first insulating layer and the second insulating layer, and the conductive rubber layer has a contact portion that is exposed from and protrudes from the detection opening;
a first buckle including:
a sheet embedded in-between the first insulating layer and the second insulating layer and being in contact with the conductive rubber layer; and
a pillar extending from the sheet and passing through the sealing hole and the thru-hole, wherein a connection between the pillar and the sealing hole of the first insulating layer has an interference-fit configuration;
a sealing gasket including:
an inner ring arranged in the thru-hole and abutting against on a part of the first insulating layer that is located outside of the sealing hole, wherein the pillar is arranged inside of the inner ring; and
an outer ring extending from the inner ring and abutting against the second surface of the wearable carrier;
a second buckle that is engaged with the pillar of the first buckle and that abuts against the outer ring so as to enable the outer ring to be sandwiched between the second buckle and the wearable carrier; and
a signal transceiver having an insulating portion and a conductive portion that is arranged inside of the insulating portion, wherein the insulating portion abuts against the outer ring of the sealing gasket, and the conductive portion is detachably connected to the first buckle and the second buckle of the signal detection device;
wherein, when the physiologic detection apparatus is worn by a user and is immersed in liquid, the contact portion of the conductive rubber layer is configured to receive a physiologic signal from the user, and the first buckle and the second buckle are configured to transmit the physiologic signal from the contact portion to the signal transceiver.

2. The physiologic detection apparatus according to claim 1, wherein the conductive rubber layer has an opening, the sheet is sandwiched between the conductive rubber layer and the second insulating layer, and the pillar sequentially passes through the opening, the sealing hole, and the thru-hole.

3. The physiologic detection apparatus according to claim 1, wherein the signal detection device includes an antistatic layer that is formed on the first surface and that is arranged adjacent to the contact portion of the conductive rubber layer.

4. The physiologic detection apparatus according to claim 1, wherein the second buckle has:
a crimping ring abutting against the outer ring; and
a fixing portion extending from an inner edge of the crimping ring and passing through the thru-hole, wherein the fixing portion is fixed to the pillar.

5. The physiologic detection apparatus according to claim 1, wherein, when the physiologic detection apparatus is immersed in the liquid, the liquid does not flow to the pillar of the first buckle.

6. The physiologic detection apparatus according to claim 1, wherein the first insulating layer, the conductive rubber layer, the second insulating layer, the first buckle, the sealing gasket, and the second buckle are jointly defined as a detection module, and wherein a quantity of the detection module is two, and the wearable carrier is elongated and includes:
an elastic adjustment segment configured to change a wearable length of the wearable carrier; and
a detection segment connected to an end of the elastic adjustment segment, the two detection modules being disposed on the detection segment;
wherein the conductive portion of the signal transceiver is detachably connected to the first buckle and the second buckle of each of the two detection modules of the signal detection device.

7. The physiologic detection apparatus according to claim 6, wherein the two detection modules are spaced apart from each other and are in a mirror symmetrical arrangement, and the first buckle, the sealing gasket, and the second buckle of one of the two detection modules are respectively arranged adjacent to the first buckle, the sealing gasket, and the second buckle of another one of the two detection modules.

8. The physiologic detection apparatus according to claim 6, wherein the detection segment has an assembling portion arranged away from the elastic adjustment segment, and the signal detection device includes:
an adjustment metal ring that is arranged between the elastic adjustment segment and the detection segment so as to enable the elastic adjustment segment to change the wearable length of the wearable carrier through the adjustment metal ring; and
a metal hook assembled to the elastic adjustment segment, wherein the metal hook is detachably engaged with the assembling portion of the detection segment so as to enable the wearable carrier to have a loop shape.

9. The physiologic detection apparatus according to claim 6, wherein the signal detection device includes a waterproof cloth disposed on the detection segment, and wherein the waterproof cloth is disposed on the second insulating layers of the two detection modules and is located between the contact portions of the two detection modules.

10. A signal detection device, comprising:
a wearable carrier having a first surface and a second surface that is opposite to the first surface, wherein the wearable carrier has a thru-hole;
a first insulating layer disposed on the first surface of the wearable carrier and having a sealing hole that corresponds in position to the thru-hole;
a conductive rubber layer disposed on the first insulating layer;
a second insulating layer disposed on the conductive rubber layer and having a detection opening, wherein the conductive rubber layer is sealed and sandwiched between the first insulating layer and the second insulating layer, and the conductive rubber layer has a contact portion that is exposed from and protrudes from the detection opening;
a first buckle including:
a sheet embedded in-between the first insulating layer and the second insulating layer and being in contact with the conductive rubber layer; and
a pillar extending from the sheet and passing through the sealing hole and the thru-hole, wherein a connection between the pillar and the sealing hole of the first insulating layer has an interference-fit arrangement;
a sealing gasket including:
an inner ring arranged in the thru-hole and abutting against on a part of the first insulating layer that is located outside of the sealing hole, wherein the pillar is arranged inside of the inner ring; and
an outer ring extending from the inner ring and abutting against the second surface of the wearable carrier;
a second buckle that is engaged with the pillar of the first buckle and that abuts against the outer ring so as to enable the outer ring to be sandwiched between the second buckle and the wearable carrier.
